(19) United States
(12) Patent Application Publication
(10) Pub. No.: US 2009/0043189 A1
(43) Pub. Date: Feb. 12, 2009

(54) Mobile combined MRI/PET apparatus

(76) Inventors: Ralf Ladebeck, Erlangen (DE); Diana Martin, Herzogenaurach (DE); Sebastian Schmidt, Weisendorf (DE)

Correspondence Address: HARNESS, DICKEY & PIERCE, P.L.C., P.O.BOX 8910, RESTON, VA, 20195, US

(21) Appl. No.: 12/222,209

(22) Filed: Aug. 5, 2008

(30) Foreign Application Priority Data
Aug. 7, 2007 (DE) 10 2007 037 102.2

(51) Int. Cl. A61B 5/05

(52) U.S. Cl. 600/411

(57) ABSTRACT

A combined positron emission tomography/magnetic resonance imaging apparatus, in a trailer housing is disclosed, for imaging organs of an examination object in an examination space. In at least one embodiment, the positron emission tomography/magnetic resonance imaging apparatus includes a positron emission tomography apparatus with at least one radiation detector and a magnetic resonance imaging apparatus with at least one main magnetic field coil for generating a main magnetic field, at least one gradient coil for generating a magnetic gradient field, and a radio-frequency antenna device for transmitting excitation pulses and receiving magnetic resonance signals from the examination space, the radiation detector and the at least one gradient coil being arranged coaxially and at substantially the same axial height. In order to create a mobile MRI/PET apparatus which is designed as compactly as possible and, firstly, satisfies the radiation protection requirements for protecting the surroundings of the measurement apparatus to the best possible extent and, secondly, excludes interfering environmental influences on the measurement apparatus, a shielding arrangement with at least one shielding element for attenuating the main magnetic field and the gradient field and also the annihilation radiation outside the trailer housing is provided in at least one embodiment.

### PRIORITY STATEMENT

The present application hereby claims priority under 35 U.S.C. §119 on German patent application number DE 10 2007 037 102.2 filed Aug. 7, 2007, the entire contents of which is hereby incorporated herein by reference.

### FIELD

Embodiments of the invention generally relate to a mobile combined MRI/PET apparatus. In particular, at least one embodiment relates to a combined positron emission tomography/magnetic resonance imaging apparatus in a trailer housing.

### BACKGROUND

In recent times, so-called hybrid modalities, such as combinations of positron emission tomography and computed tomography (PET/CT), single photon emission computed tomography and computed tomography (SPECT/CT), magnetic resonance imaging and positron emission tomography (MRI/PET), and magnetic resonance imaging and single photon emission computed tomography (MRI/SPECT), respectively, have become more important in medical imaging. In the case of these combinations, it is advantageous to combine a modality with a high spatial resolution, such as MRI or CT, with a modality with a high sensitivity, i.e. nuclear medicine methods such as SPECT or PET, abbreviated NM in the following. Some of these machines permit the simultaneous and isocentric imaging of the examination volume. Precisely in the early phase, it is not possible to use these new hybrid modalities so intensively that a continuous occupancy is ensured.

In order to ensure the cost-effectiveness of large machines such as MRI/PET or PET/CT, the system should be set up as a mobile unit. When housed in a trailer, the systems can then serve a number of clinics alternately. It is often technologically challenging to modify the machines in such a way that they can both be housed in the limited space of a trailer and that they can cope with new environmental conditions at every location. By way of example, in the case of mobile PET/CT systems, the high sensitivity to magnetic fields is a disadvantage which requires an adjustment method that is time consuming and expensive in terms of staff every time the system's location changes.

Mobile MRI systems, PET systems and PET/CT systems are known per se. In particular, the combination of MRI and PET in one mobile unit is of interest. However, the high interference sensitivity of conventional PET systems to external magnetic fields—so high that even interference by the Earth's magnetic field can influence the measurement—is disadvantageous. This means that time consuming system adjustments are required after every change of location. In the case of separate systems, indications requiring both MRI and PET examinations have to additionally resolve logistics regarding the patient and the machine.

DE 0 2005 015 070 discloses a method for imaging an examination object in an examination space by way of a combined positron emission tomography and magnetic resonance imaging scanner. The positron emission tomography scanner includes a machine part assigned to the examination space with a gamma ray detector, the detector acquiring gamma radiation emitted from the examination space by the examination object. The magnetic resonance imaging scanner includes a main magnetic field coil for generating a main magnetic field, a gradient coil system which generates magnetic gradient fields in the examination space, and a radio-frequency antenna device, which transmits excitation pulses into the examination space and/or receives magnetic resonance signals from the examination object in the examination space. A radio-frequency shield is arranged between the gradient coil system and the radio-frequency antenna device, which decouples the radio-frequency antenna device from the gradient coil system.

Furthermore, avalanche photodiode (APD) modules used in PET/MRI imaging are disclosed in WO 2006/071922 A2. Each module includes a number of independent, optically isolated detectors. Each detector includes an arrangement of scintillator crystals, which are read by a corresponding arrangement of APDs. The modules are arranged in the MRI tunnel. In this manner, the APDs can be used to record PET and MRI images with a high resolution and which are artifact free.

In addition to combining the respective modalities and miniaturizing them for installation in a spatially limited trailer, it is additionally important to firstly limit the effects of the measurement apparatus on the surroundings and, secondly, limit the influence of the surroundings on the measurement apparatus. For instance, the radiation and the magnetic field from the measurement apparatus must not harm passers-by outside the trailer (limit of cardiac pacemakers). In addition, gamma rays can penetrate outward during PET measurements. Likewise, this must not lead to a significant increase in the radioactive exposure of the surroundings of the trailer. Conversely, radiation sources or magnetic fields outside the trailer must not influence the measurement apparatus in the trailer's interior.

### SUMMARY

In at least one embodiment of the present invention, a mobile MRI/PET apparatus is disclosed which is designed as compactly as possible and in which, firstly, the radiation protection requirements for protecting the surroundings of the measurement apparatus are satisfied to the best possible extent and, secondly, interfering environmental influences are kept from the measurement apparatus.

According to at least one embodiment of the invention, a hybrid MRI/PET system, which can simultaneously and isocentrically record data from both modalities, is installed in a suitable trailer so that it can be moved from one location to another and operated there. According to at least one embodiment of the invention, the shielding of both the gamma rays and the magnetic field outside the mobile apparatus is optimized in such a way that the radiation exposure drops to a minimum outside the trailer housing. For this purpose, a ferromagnetic housing is integrated into the trailer housing, by means of which the magnetic field lines are compacted over a short distance and thus a rapid decrease in the field strength is achieved. Since the ferromagnetic housing is composed of a material with a relatively high atomic number, the interaction cross section for interaction with electromagnetic radiation is also relatively large. Thus, corresponding shielding of electromagnetic radiation is provided, i.e. γ-radiation penetrating outward during a PET measurement is absorbed here.

The arrangement of the ferromagnetic material is optimized according to the requirements of the shield for the magnetic field. This brings about a shape which is defined by the leakage field distribution (dipole field) of the magnet. On the other hand, to a first approximation, the γ-radiation uniformly distributes itself on a spherical surface and thus decreases with the square of the distance from the examination space. The shield which protects the surroundings from γ-radiation is preferably composed of lead. According to at least one embodiment of the invention, the shield is optimized in such a way with regard to its chemical composition, its arrangement around the hybrid MRI/PET system, and its thickness that it contributes to shielding the magnetic field of the MRI component. This results in the reduction of weight and savings in the cost of the shielding.

The combined positron emission tomography/magnetic resonance imaging apparatus in a trailer housing for imaging organs of an examination object in an examination space according to at least one embodiment of the invention comprises: a positron emission tomography apparatus with at least one radiation detector for acquiring positron annihilation radiation from the examination space and a magnetic resonance imaging apparatus with at least one main magnetic field coil for generating a main magnetic field in the examination space, at least one gradient coil for generating a magnetic gradient field in the examination space, and a radio-frequency antenna device for transmitting excitation pulses into the examination space and receiving magnetic resonance signals from the examination space, the radiation detector and the at least one gradient coil being arranged coaxially and at substantially the same axial height around the examination space, and is characterized by a shielding arrangement with at least one shielding element for attenuating the main magnetic field and the gradient field and also the annihilation radiation outside the trailer housing.

In this case, the at least one shielding element of the shielding arrangement is preferably ferromagnetic. In this manner, the magnetic field can be influenced in the most effective way.

Furthermore, the at least one shielding element has a greater wall thickness where there is no metal between the shielding element and the examination space to shield γ-radiation. This results in the shielding element being optimized with regard to weight and cost.

In a further example embodiment of the invention, the at least one shielding element comprises materials with a high atomic number and in particular Co for attenuating the annihilation radiation. In this manner, both the magnetic field outside the trailer and the electromagnetic radiation are attenuated by one and the same material.

In particular, at least two shielding elements of the shielding arrangement are arranged symmetrically with respect to the one magnetic resonance imaging apparatus. As a result, during the set-up of the trailer with the combined positron emission tomography/magnetic resonance imaging apparatus not only is the radiation exposure attenuated in a particular direction, but also this occurs symmetrically with regard to the MRI/PET apparatus.

In another example embodiment of the invention, the positron emission tomography apparatus can be removed from the magnetic resonance imaging apparatus. This results in the possibility of increased patient comfort in the magnetic resonance imaging apparatus by removing the positron emission tomography apparatus.

In this embodiment, the positron emission tomography apparatus can, in particular, be interchanged between different magnetic resonance imaging apparatuses. This is advantageous if the combined positron emission tomography/magnetic resonance imaging apparatus is equipped with a plurality of magnetic resonance imaging apparatuses. In the most extreme case, it is even possible, in this manner, for the positron emission tomography apparatus to be brought to predetermined target locations and be operated in correspondingly equipped MRI systems, if required.

In order to be able to achieve this, it is necessary for the positron emission tomography apparatus to be adaptable to differently designed MRI systems. For this purpose, adapter attachments in particular are provided for adapting the positron emission tomography apparatus to the different tunnel designs of the magnetic resonance imaging apparatuses.

So that the combined positron emission tomography/magnetic resonance imaging apparatus can be set up independently of magnetic fields of the surroundings, such as the Earth's magnetic field, it is preferable for the positron emission tomography apparatus to comprise avalanche photodiodes for verifying γ-radiation. It is thus possible to set-up the trailer with the mobile combined positron emission tomography/magnetic resonance imaging apparatus with an almost arbitrary orientation.

One of the many advantages of the apparatus according to at least one embodiment of the invention is that only the spatial requirements of an MRI system have to be provided in the trailer since the PET component is fully integrated. Only minimal spatial requirements for the specific PET electronics, which can be housed in the machine room next to the MRI components, arise. All other components, such as, for example, processor, console, patient couch, power supply, and cooling, are jointly designed for both partial modalities. In contrast to known hybrid modalities such as PET/CT, the partial modalities are in this case arranged not one behind the other but nested one within the other, optimizing the space requirement which is particularly limited in trailer surroundings.

Moreover, the use of semiconductor technology, such as, for example, avalanche photodiodes, rather than the photomultiplier technology conventional in PET, achieves immunity to interference by magnetic fields. This technology permits simple change of location of the mobile PET or MRI/PET system in a suitable trailer, i.e. the mobile PET or MRI/PET system can be set up quickly and without problems at the respectively desired location without having to undertake any additional preliminary measures. In particular, the use of avalanche photodiodes (APD) in the PET apparatus removes the dependence on the Earth's magnetic field, i.e. the orientation of the trailer during its set-up is not critical.

### BRIEF DESCRIPTION OF THE DRAWINGS

Further features and advantages of the apparatus according to the invention emerge from the following description of example embodiments with reference to the attached drawing, in which

FIG. 1 schematically shows a combined PET/MRI apparatus according to the prior art in a perspective illustration,

FIG. 2 schematically shows a combined PET/MRI apparatus according to the prior art in a side view,

FIG. 3 schematically shows a combined PET/MRI apparatus with equipotential lines of the generated magnetic field in and around a trailer from above,

FIG. 4 schematically shows a combined PET/MRI apparatus with equipotential lines of the generated magnetic field in and around a trailer from above if a shielding device is provided,

FIG. 5 schematically shows a combined PET/MRI apparatus with equipotential lines of the generated magnetic field in and around a trailer from the side,

FIG. 6 schematically shows a combined PET/MRI apparatus with equipotential lines of the generated magnetic field in and around a trailer from the side if a shielding device is provided,

FIG. 7 schematically shows a combined PET/MRI apparatus with equipotential lines of the generated magnetic field in and around a trailer from the front, and

FIG. 8 schematically shows a combined PET/MRI apparatus with equipotential lines of the generated magnetic field in and around a trailer from the front if a shielding device is provided.

The figures are not to scale. Similar or similarly-acting elements are provided with the same reference symbol.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

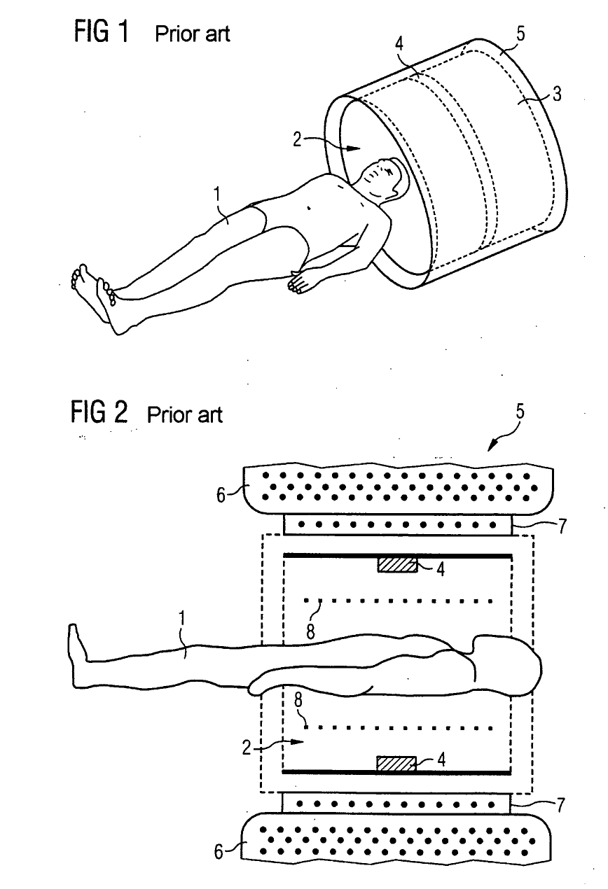

In the case of combined PET and MRI, an examination object 1 is put into an examination space 2, as illustrated in FIG. 1. This examination space 2 is surrounded by a PET apparatus 3 with a detector device 4. The detector device 4 is generally an arrangement of scintillation crystals (not shown), arranged annularly around the examination space 2. Photons with an energy of 511 keV (annihilation radiation of positrons) are converted into light quanta in the scintillation crystals, which in turn are led, preferably via optical waveguides (not shown), to photodetectors (not shown) which generate electrical output signals depending on the number of light quanta.

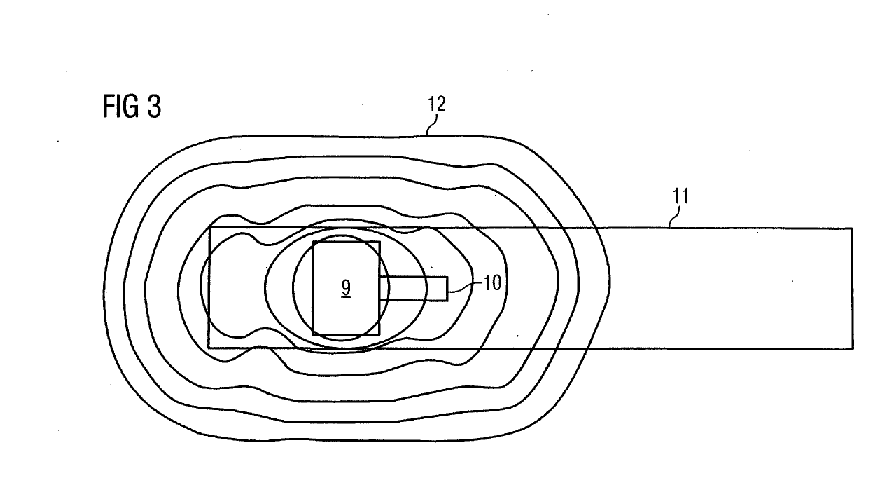

In order to improve the spatial resolution of the examination of the examination object 1, the PET apparatus is surrounded by an MRI apparatus 5. This substantially comprises a gradient coil 7 and a radio-frequency antenna device 8 in addition to a basic field magnet 6. These elements are explained below with reference to FIG. 2.

FIG. 2 illustrates such a construction with further details in cross section. The examination object 1 is partly located within the examination space 2. The coil 6 for generating a main magnetic field is arranged in the outermost position around the examination space 2. The magnetic field generated by the coil 6 in the examination space 2 has an axis which corresponds to the main axis of the examination object 2 in the plane of the image.

Within the coil 6, the gradient coil 7 is arranged as a further coil by means of which a gradient field is generated in the examination space 2. The gradient coil 7 is wedged in or screwed to the basic magnetic field coil 6 so that the two coils 6 and 7 are fixedly connected to one another.

A radio-frequency electromagnetic field is radiated into the examination space 2 by means of a radio-frequency antenna device 8 which is a part of the MRI apparatus.

FIG. 3 now shows, from the top, a combined positron emission tomography/magnetic resonance imaging apparatus as a unit 9 with a patient couch 10 installed in a trailer 11. In particular, this trailer 11 can be a high capacity trailer of a truck, the combined positron emission tomography/magnetic resonance imaging apparatus 9 preferably being positioned directly above the axle or axles of the trailer due to its weight. To clarify the fields generated by the measurement apparatus, equipotential lines 12, which schematically reproduce the extent of the magnetic field, are illustrated in FIG. 3.

As can be seen, the strongest field strength is located in the direct vicinity of the measurement apparatus 9. The field strength decreases with increasing distance from the measurement apparatus 9. In this case, the profile of the equipotential lines 12 is determined by the materials in the surroundings of the measurement apparatus 9. The field originating from the measurement apparatus 9 extends further into the surroundings where there are no magnetically relevant materials in the vicinity of the measurement apparatus 9. Where there are materials which influence the field in the vicinity of the measurement apparatus, the field does not extend as far into the surroundings. The outermost illustrated equipotential lines 12 in FIG. 3 thus form an oval, in which the major axis runs parallel to the longitudinal axis of the trailer.

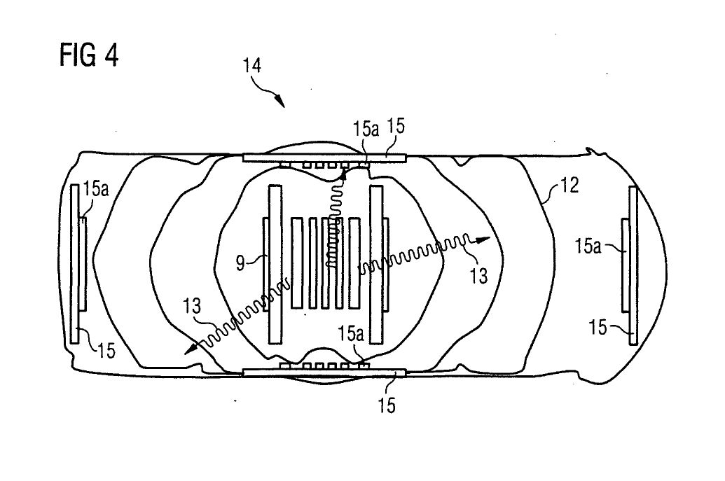

According to an embodiment of the invention, the profile of the equipotential lines 12 is changed in a targeted manner, in order to minimize the exposure of the surroundings to the magnetic field. This is schematically illustrated in FIG. 4.

FIG. 4 shows the measurement apparatus 9 in the trailer with further details in a plan view. The measurement apparatus 9 has a plurality of coils surrounding the examination space 2, the coils being arranged, one behind the other, along the longitudinal axis of the trailer, and illustrated as black bars in FIG. 4. In order to limit the field extending transversely to the longitudinal axis of the trailer as far as possible, a shielding apparatus 14 is provided in the trailer 11, which at least partly surrounds the measurement apparatus 9. The shielding apparatus 14 accordingly comprises a plurality of shielding elements 15 which are preferably arranged symmetrically with respect to the measurement apparatus 9, so that the field strength is reduced evenly in the vicinity of the measurement apparatus 9.

As can be seen by comparing FIG. 3 and FIG. 4, the arrangement of the shielding elements 15 at the critical positions around the measurement apparatus 9 leads to a considerable decrease of the field outside the trailer. Such a decrease of the field is particularly desirable in the surroundings to the side of the trailer, since passers-by come particularly close to the measurement apparatus 9 in the interior of the trailer, possibly without even noticing this. However, in order to reduce the radiation exposure in the direction of travel of the trailer 11, it is of course possible to arrange further shielding elements 15 in front of and behind the measurement apparatus 9. This is likewise illustrated in FIG. 4. Here, too, this results in a corresponding reduction in the field strength. Overall, the oval profile of the field strength around the measurement apparatus 9 is substantially maintained; however, the extent of the field is mainly limited to the interior of the trailer 11 only.

The fact that this effect holds for both the magnetic field 12 and the electromagnetic radiation is indicated by rays 13, which are intended to represent gamma quanta escaping the examination space. These rays 13 are also shielded by the shielding apparatus 14 with elements 15, so that they cannot, or can only in a smaller proportion, reach the surroundings from the trailer 11. It is substantially only where the elements 15 are at a greater distance from the examination space that the gamma quanta can escape from the apparatus. In other words, in the illustration in FIG. 4, the two shielding elements 15, which are situated transverse to the apparatus 9 and are located close to it, are particularly effective since they significantly reduce the free solid angle for the γ-radiation. In this case, what must be taken into consideration is that the shield for the γ-radiation, as a part of the PET component, can be thinner where magnetic iron is present as part of the MRI component. In other words, the shield for the magnetic field and the shield for the γ-radiation complement each other if they are composed of a magnetic material with a high atomic number, as is proposed according to an embodiment of the invention.

In order to achieve both shielding of the magnetic field and shielding from ionizing electromagnetic radiation, suitable materials must be used for the shielding elements 15. For this purpose, ferromagnetic materials with a high atomic number Z are particularly suitable. In particular, these are ferromagnetic Fe and Co alloys.

A further improvement in the shielding of γ-radiation is achieved by increasing the wall thickness of the respective shielding elements 15 at those locations where it appears to be necessary. As shown in FIG. 4, the shielding element 15 in this case has a thicker wall thickness at those locations where there is no metal shielding the γ-radiation between the shielding element 15 and the examination space 2.

In FIG. 4, these wall reinforcements 15a are reinforcement ribs on the shielding elements 15 which are applied where metal does not surround the examination space 2, that is to say where there is no coil or no iron brace. The ribs 15a on the shielding elements 15 to the side are thus arranged in a complementary fashion to the schematically illustrated metal-containing structures of the apparatus 9. In the case of the shielding elements 15 at the top end and bottom end of the apparatus, the wall in the section 15a is not reinforced by ribs but by a whole reinforcement plate. In this manner it is ensured that, in addition to the attenuation of the magnetic field, there is also a decrease in the radioactive radiation.

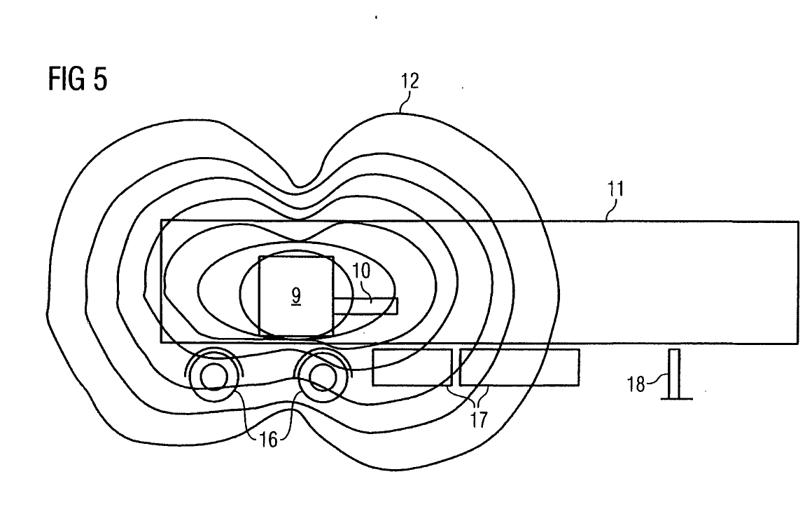

The further FIGS. 5 to 8 illustrate the equipotential lines 12 in a side view, and in a front or back view of the trailer 11. The weight distribution of the measurement apparatus 9 with respect to the axles 16 of the trailer 11 is apparent in FIG. 5. Due to its large weight, the measurement apparatus 9 with the patient couch 10 is preferably arranged over the two axles 16; the trailer can then be parked with the aid of a support apparatus 18. Supply devices 17 outside the trailer 11 serve to house material and energy sources outside the interior, in particular if the material is to be stored in particular conditions, or the energy sources, such as compressors and the like, would burden the staff in the interior of the trailer.

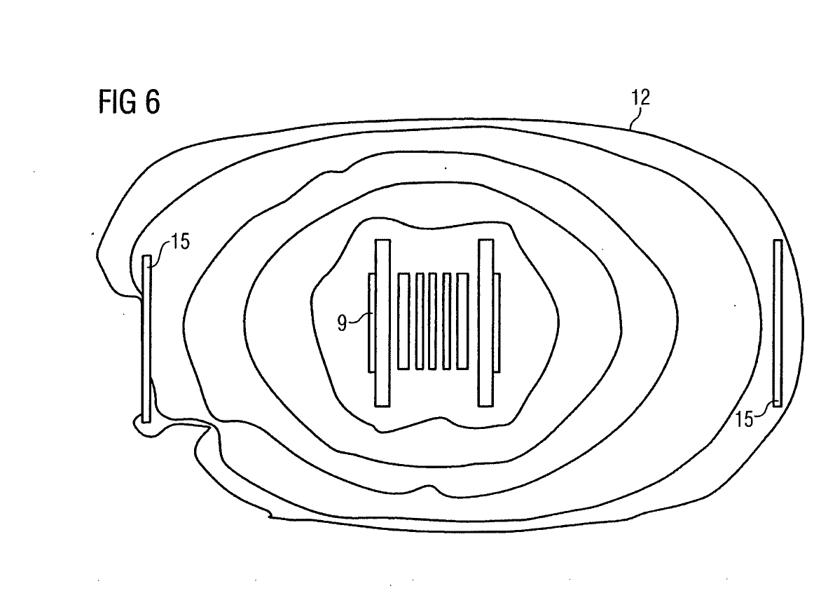

FIG. 6 shows the profile of the equipotential lines 12 when using shielding elements 15. Whereas the field is geometrically limited in the longitudinal direction of the trailer 11, it is virtually uninfluenced downward and upward. This is also not required in the case of the trailer according to FIG. 5. In order to reduce the weight of the trailer 11, only the two shielding elements shown are provided in the embodiment according to FIG. 6, since the shielding elements 15 comprising a ferromagnetic material with a high atomic number Z contribute to the weight.

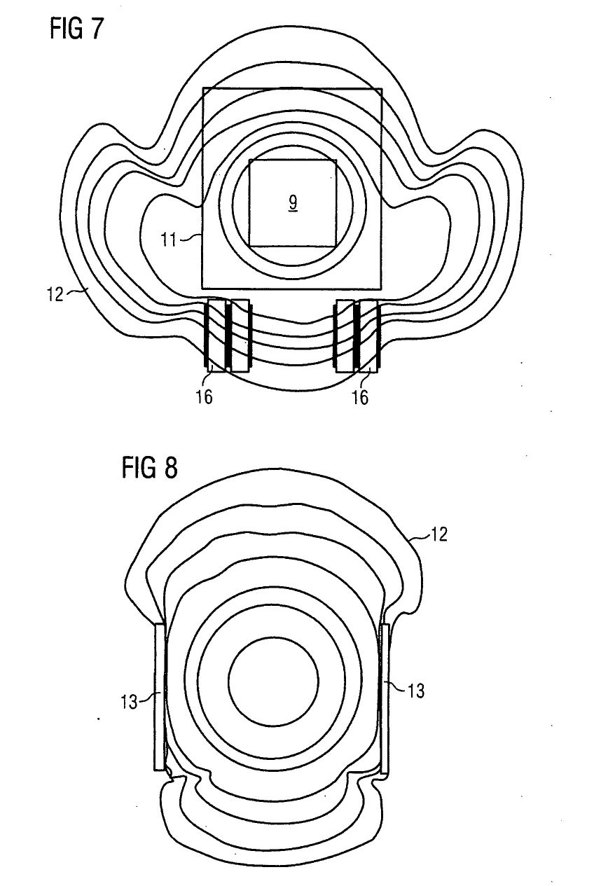

Finally, FIGS. 7 and 8 show the profile of the equipotential lines 12 without or with shielding elements 12. As is the case in the other figures, FIG. 7 shows the outline of the trailer 11 and the measurement apparatus 9 without shielding. In contrast, for the sake of clarity FIG. 8 only shows the profile of the field. Whereas the magnetic field in FIG. 7 shows a significant extent to the side of the trailer 11, this extent has virtually disappeared in the embodiment in FIG. 8 thanks to the shield. The effectiveness of the shield can thus be seen.

Furthermore, in the following text, some advantages and features of mobile combined positron emission tomography/magnetic resonance imaging are explained.

In an example embodiment (not shown), the PET component in the case of mobile combined positron emission tomography/magnetic resonance imaging is designed as an “insert”, i.e. as a flexible insertion into the MRI system of the mobile combined positron emission tomography/magnetic resonance imaging. The infrastructure for the PET system, such as processor, electronics, supply, etc., is present in this case, so that there is no need for retrofitting. However, the PET component can be removed to provide a larger MRI tunnel, which improves patient comfort. Depending on requirements, the removable PET system is positioned in the trailer 11, or can be interchanged between different mobile or fixedly installed MRI systems if the appropriate infrastructure is present.

In particular, the PET component can thus serve a plurality of (fixedly installed) MRI systems. In this case, the PET component is brought to the respective target locations in a truck, and can be operated, if required, in correspondingly equipped MRI systems.

In a further example embodiment, the PET system is provided with suitable adapter attachments. Differences in the tunnel designs of the MRI systems can thereby be evened out. Preferably, the infrastructure for the PET component is provided in the MRI system. In this embodiment, the PET component can be designed independently of field strength, so that the PET component can be flexibly interchanged between MRI systems with different field strengths.

Due to the mobile design of the hybrid MRI/PET system, its use can be optimized. According to an example embodiment of the invention, in order to allow a mobile embodiment, the spatial requirements of the hybrid MRI/PET system are reduced to an extent permissible in a trailer and the radiation exposure is minimized in the surroundings of the trailer. According to an example embodiment of the invention, this is achieved by technical measures such as the concentric integration of the partial modalities and the combined use of central components. Here, the use of semiconductor technologies allows a reduction of the set-up time, which in turn increases net usage time. Due to the alternative use of the PET component, the flexibility is increased, and the trailer systems or fixedly installed systems can be equipped depending on requirements.

Since the combined positron emission tomography/magnetic resonance imaging apparatus furthermore includes avalanche photodiodes in the positron emission tomography apparatus 3 for verifying γ-radiation, it can be set up at with an arbitrary orientation with respect to the surrounding (Earth's) magnetic field.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDS; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A combined positron emission tomography/magnetic resonance imaging apparatus in a trailer housing for imaging organs of an examination object in an examination space, comprising:
a positron emission tomography apparatus, including at least one radiation detector to acquire positron annihilation radiation from the examination space;
at least one magnetic resonance imaging apparatus including
at least one main magnetic field coil to generate a main magnetic field in the examination space,
at least one gradient coil to generate a magnetic gradient field in the examination space, and
a radio-frequency antenna device to transmit excitation pulses into the examination space and receive magnetic resonance signals from the examination space, the radiation detector and the at least one gradient coil being arranged coaxially and at substantially the same axial height around the examination space; and
a shielding arrangement including at least one shielding element, to attenuate the main magnetic field, the gradient field and the annihilation radiation outside the trailer housing.

2. The combined positron emission tomography/magnetic resonance imaging apparatus as claimed in claim 1, wherein the at least one shielding element of the shielding arrangement is ferromagnetic.

3. The combined positron emission tomography/magnetic resonance imaging apparatus as claimed in claim 1, wherein the at least one shielding element includes a greater wall thickness where there is no metal between the shielding element and the examination space to shield γ-radiation.

4. The combined positron emission tomography/magnetic resonance imaging apparatus as claimed in claim 1, wherein the at least one shielding element includes materials with a high atomic number to attenuate the annihilation radiation.

5. The combined positron emission tomography/magnetic resonance imaging apparatus as claimed in claim 1, wherein at least two shielding elements of the shielding arrangement are arranged symmetrically with respect to the one magnetic resonance imaging apparatus.

6. The combined positron emission tomography/magnetic resonance imaging apparatus as claimed in claim 1, wherein the positron emission tomography apparatus is removable from the magnetic resonance imaging apparatus.

7. The combined positron emission tomography/magnetic resonance imaging apparatus as claimed in claim 6, wherein the at least one magnetic resonance imaging apparatus includes a plurality of magnetic resonance imaging apparatuses, in which the positron emission tomography apparatus is interchangeable between different magnetic resonance imaging apparatuses.

8. The combined positron emission tomography/magnetic resonance imaging apparatus as claimed in claim 7, further comprising:
adapter attachments to adapt the positron emission tomography apparatus to different tunnel designs of the magnetic resonance imaging apparatuses.

9. The combined positron emission tomography/magnetic resonance imaging apparatus as claimed in claim 1, wherein the positron emission tomography apparatus includes avalanche photodiodes for verifying γ-radiation.

10. The combined positron emission tomography/magnetic resonance imaging apparatus as claimed in claim 2, wherein the at least one shielding element includes a greater wall thickness where there is no metal between the shielding element and the examination space to shield γ-radiation.

11. The combined positron emission tomography/magnetic resonance imaging apparatus as claimed in claim 4, wherein the at least one shielding element includes Co to attenuate the annihilation radiation.

12. The combined positron emission tomography/magnetic resonance imaging apparatus as claimed in claim 7, wherein the positron emission tomography apparatus includes avalanche photodiodes for verifying γ-radiation.
